(19) United States
(12) Plant Patent Application Publication
(10) Pub. No.: US 2016/0330890 P1
(43) Pub. Date: Nov. 10, 2016

(54) Hydrangea plant named 'BC7.8'

(50) ;

(71) Applicant: Bay City Flower Company, Half Moon Bay, CA (US)

(72) Inventor: Harrison M. Higaki, San Mateo, CA (US)

(21) Appl. No.: 14/545,430

(22) Filed: May 4, 2015

(51) Int. Cl. A01H 5/00

(52) U.S. Cl. PLT/250

(57) ABSTRACT

A new and distinct cultivar of Hydrangea macrophylla (Thunb.) named ‘BC7.8’ originated as a controlled cross between U.S. Pat. No. 18,592, named ‘True Blue’ and the commercial variety ‘LK49’. The cultivar ‘BC7.8’ can be blue or red depending on the acidity of the soil and the presence of aluminum. The variety ‘BC7.8’ has relatively large, attractive inflorescences with relatively large sepalous florets, distinct sepal pigmentation and good commercial characteristics. When grown without aluminum and under soil conditions to create pink flowers, the upper sides of the sepals are R.H.S. 70 C (red-purple group) and the under sides of the sepals are R.H.S. 65 C (red-purple group).

### BOTANICAL CLASSIFICATION

Hydrangea macrophylla (Thunb.) ‘BC7.8’

### VARIETY DENOMINATION

‘BC7.8’

### BACKGROUND OF THE INVENTION

This invention relates to a new and distinct cultivar of the Saxifragaceae family. The botanical name of the plant is Hydrangea macrophylla (Thunb.) ‘BC7.8’.

The new cultivar originated as a seedling from a controlled cross between Hydrangea macrophylla (Thunb.) ‘True Blue’—U.S. Plant Pat. No. 18,593 which was the seed parent, and the unpatented variety known as ‘LK49’ which was the pollen parent. ‘LK49’ is relatively compact plant with wiry stems, relatively small leaves, relatively small sepalous florets, and inflorescences that are resistant to being damaged by conditions in commercial coolers.

The variety ‘BC7.8’ has relatively large inflorescences, relatively large sepalous florets, uniform sepal pigmentation at maturity and good commercial characteristics. The variety ‘BC7.8’ has pigmented sepals, and can be grown in soil conditions treated with aluminum to produce blue pigmentation, which is not described here. The color of the sepals changes as the plant ages. Below is a table comparing the new variety to similar varieties.

TABLE 1

| | | Commercial | | |
|---|---|---|---|---|
| | | variety | | |
| | | “Venedig’ | | |
| | | which may be | | |
| | | U.S. Plant Pat. | U.S. Plant | Commercial |
| | | No. 10,928 | Pat. No. | variety |
| | New Variety | ‘Venice | 18,593 | known as |
| | ‘BC7.8’ | Raven’ | ‘True Blue’ | ‘LK49’ |
| Leaf size | 11cm × 15 cm | 12 cm wide × | | Medium |
| | | 15 cm long | | small |
| Plant | 18″ in | 15″ in | 17″ high in | |
| height | 6″ pot. | 6″ pot. | 4″ pot | |
| Stem | Strong | Stems are | Strong | Wiry |
| strength | | strong but | | |
| | | benefit from | | |
| | | being staked | | |
| Sepal | Upper side | Upper side of | Both sides | Medium |
| Pigmen- | of sepals is | sepals is | of sepals are | Dark |
| tation | R.H.S. | R.H.S. 86 A | R.H.S. 100 D | Pink-ob- |
| | 70 C (red- | (violet group); | (blue group) | served con- |
| | purple group); | Under side | at maturity | trols grown |
| | Under side | of sepals is | | alongside |
| | of sepals is | R.H.S. 88 D | | new variety. |
| | R.H.S. | (violet group) | | |
| | 65 C (red- | | | |
| | purple group) | | | |
| Sepalous | 70 mm | 50 mm to | 50 mm to | Medium |
| Floret Size | | 80 mm | 60 mm | Small- |
| | | | | observed con- |
| | | | | trols grown |
| | | | | alongside |
| | | | | new variety |

The new cultivar ‘BC7.8’ has been successfully asexually reproduced under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in Half Moon Bay, Calif. show that the combination of characteristics as herein disclosed for ‘BC7.8’ remains firmly fixed through three generations.

### DESCRIPTION OF THE DRAWINGS

The accompanying drawings consist of color photographs that show the typical plant form, including the inflorescence, foliage, and sepals.

FIG. 1 is a side view of the entire plant showing its form, growth habit, dark green foliage, inflorescence, and the color of its sepals.

FIG. 2 is a top view of the entire plant showing its form, growth habit, dark green foliage, inflorescence, and the color of its sepals.

FIG. 3 is a close-up view of the inflorescence.

FIG. 4 is a close-up top view of the upperside of a mature sepalous flower of the new variety.

FIG. 5 is a close-up side view of a mature sepalous flower of the new variety.

FIG. 6 is a close-up view of the adaxial surface of a mature leaf.

FIG. 7 is a close-up view of the base of the stem.

FIG. 8 is a close-up view of the inside of the inflorescence.

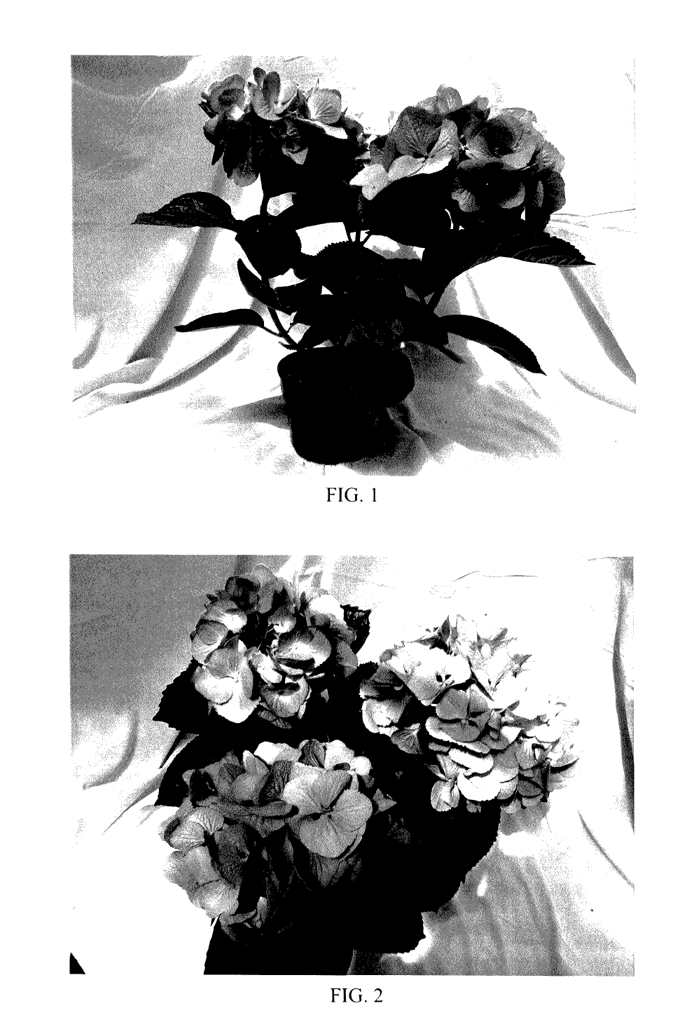

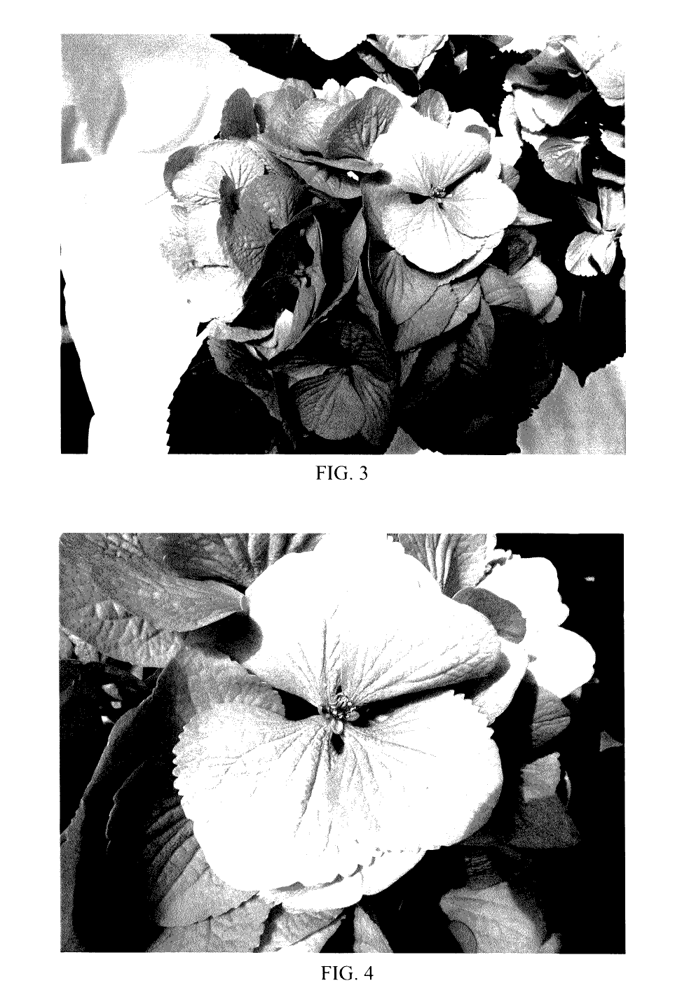

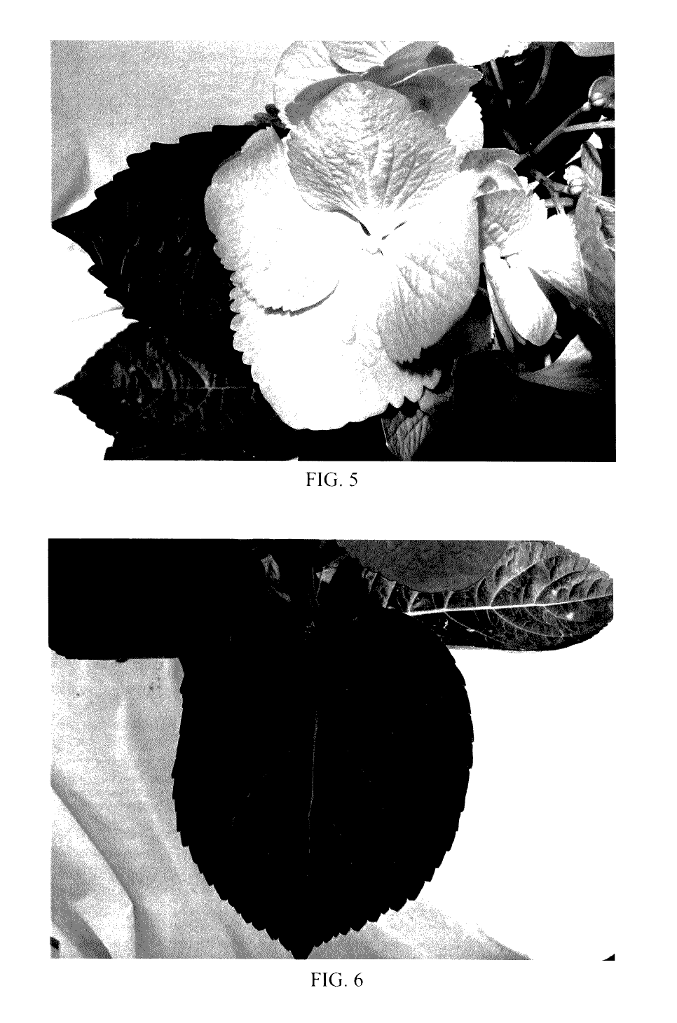

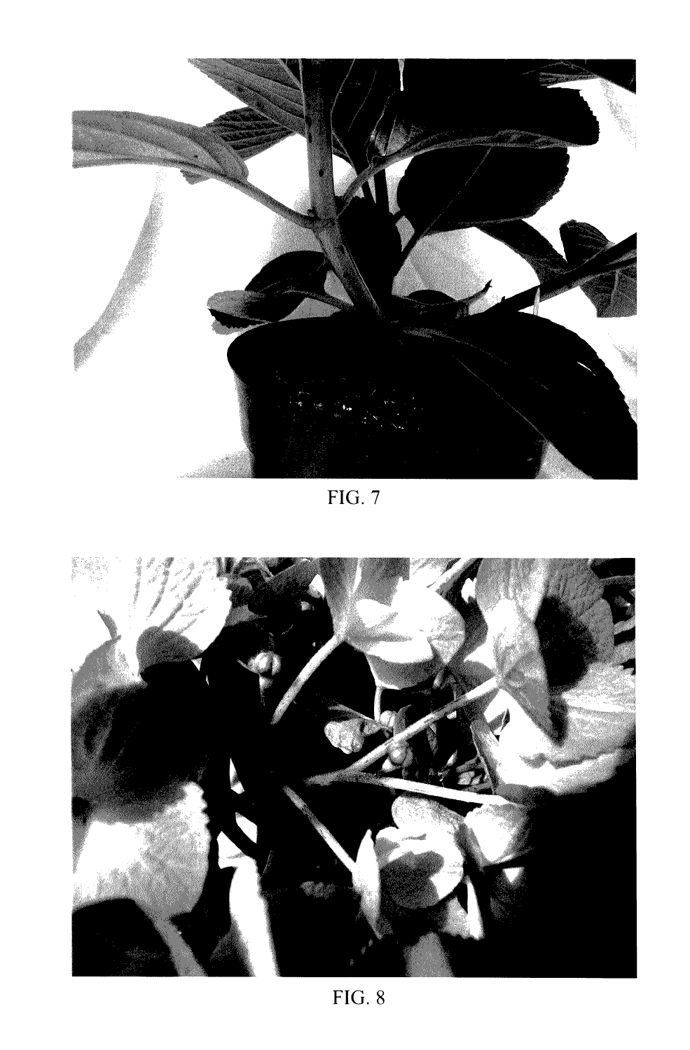

### DESCRIPTION OF THE NEW PLANT

The plant shown in the figures is approximately a year old. The plant started out as cuttings, taken from the stem of a grown plant.

‘BC7.8’ has not been observed under all possible environmental conditions. The phenotype may vary significantly with variations in environment such as temperature, light intensity and day length. Color determinations were made with The Royal Horticultural Society (R.H.S.) Colour Chart, in association with the Flower Council of Holland, located in Lieden.
The plant:
   Origin.—Controlled cross. The new cultivar originated as a seedling from a controlled cross between Hydrangea macrophylla (Thunb.) True Blue — U.S. Plant Pat. No. 18,593 which was the seed parent, and the commercial variety known as ‘LK49’ which was the pollen parent.
   Form.—Upright, compact shrub. A typical plant with a mature inflorescence that is ready for sale is approximately 18″ high and has a diameter of 18″,when grown in a 6″ pot with appropriate soil amendments.
   Growth.—Upright, vigorous growth habit. Inflorescence is large. The plant branches easily with shoots forming at the base of the plant. Lateral branches are similar in appearance and form to the main stems.
   Stems.—Lenticels are present. Lenticels are R.H.S. 86 A (violet group) and are 1 to 2 mm long. The surface of young stems is glabrous. Stems become woody as they age. The color of typical young stems and young lateral branches is R.H.S. 144 A (green group). The older portions of the stems are R.H.S. 199 B (grey-brown group). Younger portions of the stems are 8 mm in diameter. Older portions of the stems are 8 mm in diameter.
   Foliage.—Abundant. Leaves are opposite on stem and lateral branches. Shape of leaf. — Elliptic with acute base and apex. Margins are serrate. Texture. — Glabrous; veins dominate on the underside of the leaf and are sunken on the upper leaf surface. Color. — Mature leaves have an upper side that is R.H.S. 147 A (yellow-green group), and an under side that is R.H.S. 138 B (green group). Leaves are pinnately veined. The midvein and veins branching off the midvein are large and prominent on the underside of the leaves. Veins are R.H.S. 138 B (yellow-green group). Leaves are as wide as 11 cm and 15 cm long. Petioles are smooth and approximately 2.0-3.0 cm long and 4 mm wide. Petioles are R.H.S. 138 B (yellow-green group).
Buds:
   Form.—Globose with 4 to 5 connate petals. Most buds, whether they will mature into sepalous or non-sepalous florets, have 4 petals. Buds in the center of the inflorescence are non-sepalous. The majority of buds will develop into sepalous florets. They are approximately 1 mm by 1 mm when very young. Buds can be 4 mm in diameter and still unopened. Color of mature buds is R.H.S. 78 B (red-purple group).
   Aspect.—Smooth.
   Arrangement.—Borne on branched panicles.
Inflorescence:
   Form.—Paniculate. Terminal. As many as 100 individual flowers (florets) per inflorescence. Both sepalous florets and non-sepalous florets borne on the same panicle. Flowers do not produce a fragrance. The peduncle for the inflorescence is strong and upright. Very few non-sepalous florets developing except for the first on individual cymes with the subsequent three or four florets on that cyme being sepalous. Florets, both sepalous and non-sepalous, have anthers and style. Inflorescences are long-lasting, up to six weeks.
   Size of inflorescence.—Large and globose. Individual inflorescence size is dependent on the .number of florets. A typical inflorescence can grow as large as 9″ in diameter, and 5″ high.
   Shape.—Clusters of numerous small florets; sepalous florets are flat and overlap one another. Sepals are persistent.
   Appearance.—Showy.
Florets:
   General.—The non-sepalous florets at the center of the inflorescence open first. Sepalous and non-sepalous florets are perfect and complete. Corolla: Generally, for both sepalous and non-sepalous florets there are 5 petals which fall off as flower matures. Petals are typically 4 mm long and 3 mm wide. Petals are R.H.S. 78 B (red-purple group). Lenticels are present on pedicels, lenticels are not even 1 mm long and thin in diameter. Lenticels are R.H.S. 59 B (red-purple group). Pedicel length for non-sepalous florets averages 4 mm. Pedicel length of sepalous florets is approximately 30 to 40 mm in length for plants of this age. Pedicels of both sepalous and non-sepalous florets continue to elongate as the inflorescence ages. Pedicels are R.H.S. 64 C (red-purple group) when mature.
   Stamens.—8 to 10 stamens. Pollen is R.H.S. 155 C (yellow-white group). Plant produces abundant pollen. Filament is approximately 3 to 4 mm long. Filament is R.H.S. 77 B (purple group). Anther is 1 mm long and is regular and basally attached.
   Stigma.—Two to three style each, although most florets have two style. Each style has one stigma. Style is typically 2 mm long. Style is R.H.S. 77 B (violet group). Stigma is R.H.S. 65 C (red-purple group).
   Ovary.—Ovary is partially inferior.
   Sepalous florets.—General. — Veins dominate on the underside of the sepals. Number of sepals. — 4 or 5 sepals per floret, usually 4. Aspect of sepals. — Smooth and glaucescent. Shape of sepals. — Reniform with acuminate apex. Edges often wavy when the floret is young. Size of sepals. — As the florets mature, the sepals enlarge and overlap each other more and more, until, often, there is no space between the sepals when the petals of the florets open. Sepals at maturity typically range from 4 cm long and 4 cm wide to 5.5 cm wide and 5 cm long. Flowers are typically 5 to 8 cm in diameter. The uppersides of the sepals are R.H.S. 70 C (red-purple group) and the undersides are R.H.S. 65 C (red-purple group). Pigmentation develops at the tips of the sepals and travels inward towards base of the sepals.
   Fruit.—none.
   Disease and pest resistance.—unknown.

I claim:

1. A new and distinct Hydrangea macrophylla plant named ‘BC7.8’ substantially as herein shown and described.
